(19) United States
(12) Patent Application Publication
(10) Pub. No.: US 2015/0088021 A1
(43) Pub. Date: Mar. 26, 2015

(54) VITAL SIGNS SENSING APPARATUS AND ASSOCIATED METHOD

(71) Applicant: INDUSTRIAL TECHNOLOGY RESEARCH INSTITUTE, Chutung (TW)

(72) Inventors: Kuo-Hua Tseng, New Taipei City (TW); Chang-Ho Liou, Shetou Township (TW); Wen-Ching Ko, Kaohsiung City (TW)

(73) Assignee: INDUSTRIAL TECHNOLOGY RESEARCH INSTITUTE, Chutung (TW)

(21) Appl. No.: 14/142,005

(22) Filed: Dec. 27, 2013

(30) Foreign Application Priority Data
Sep. 26, 2013 (TW) 102134885

(51) Int. Cl. A61B 7/04; A61B 7/00

(52) U.S. Cl. CPC A61B 7/04; A61B 7/008; A61B 7/003; A61B 7/006
USPC 600/528; 600/586

(57) ABSTRACT

A vital signs sensing apparatus includes a sound sensing unit and a pressure unit. The sound sensing unit senses a sound inside a body of a user and produces an audio signal. The pressure unit produced a pressure signal. The pressure signal indicates a degree of closeness between the vital signs sensing apparatus and the user. The audio signal is transformed into a processed audio signal according to the pressure signal.

This application claims the benefit of Taiwan Patent Application No. 102134885, filed Sep. 26, 2013, the subject matter of which is incorporated herein by reference.

### TECHNICAL FIELD

The disclosure relates to a sensing apparatus and an associated method, and relates to a vital signs sensing apparatus and a sensing method of thereof.

### BACKGROUND

In general, the conventional stethoscope is used to listen to the sound inside the ribcage of a patient. The stethoscope is a quick, safe and convenient tool to diagnose the cardiopulmonary diseases. For a doctor in medical diagnosis, the use of a stethoscope to do auscultation of patient's ribcage can provide convenient and correct diagnosis. That is, doctors can quickly screen for patients with cardiopulmonary disease by a stethoscope.

Please refer to FIG. 1. It shows a schematic diagram of a conventional electrical stethoscope. The electrical stethoscope comprises a sound collecting unit 110, a sound hearing unit 130, and a conducting unit 120. The sound collecting unit 110 comprises several basic-function control buttons for controlling the volume of the vital signs that are received by the sound collecting unit 110. Besides, the conducting unit 120 comprises an advanced controlling panel 115 for controlling the received vital signs of the electrical stethoscope 100. For example, the advanced controlling panel 115 may be used for recording, playing or transferring the vital signs.

When a doctor uses an electrical stethoscope to make a medical diagnosis, the sound hearing unit 130 is put in the ear, and the sound collecting unit 110 is in contact with the patient's body. By controlling the volume of the sound collecting unit 110, the doctor can clearly hear heart sounds, lung sounds, and etc. By using the recording function of the advanced controlling panel 115, the vital signs can be recorded and played repeatedly, and the vital signs can be stored in the database of anamnesis.

With the aging of the population, the health warning system and the health care product are gradually developed for the future trends. For early discovering diseases, various kinds of wearable vital signs sensing apparatuses have been introduced into the market. These vital signs sensing apparatuses can continuously record and transmit heart sounds or lung sounds to the recording device and the displaying device at the backend. Doctors can analyze the vital signs by using the recording device and the displaying device and provide health warning and care solutions. The health warning and care solutions may be applied to home care, walking around care, industrial safety and health care, voluntary health warning, and etc.

Please refer to FIG. 2, which shows a schematic diagram of a system and method for tracking a vital-signs monitor patch. The monitor patch 220 is adhered to the patient's body. Besides, the monitor patch 220 comprises electrodes and various sensors for sensing the vital signs of the body. The measured vital signs can be transmitted from the monitor patch 220 to a server 260 through a bridge 240 by wireless communication.

Basically, when the electrical stethoscope 100 is used to make a medical diagnosis, the sound collecting unit 110 may be contacted with the patient's ribcage and temporally pressed by hand in order to hear the most clear vital signs.

However, the conventional vital signs sensing apparatus, for example the monitor patch 220 shown in FIG. 2, is affixed to the patient's body by adhesive tape for long time monitoring. When the body twists or sweats, the monitor patch 220 may be loosen or fall down. At that time, the monitor patch 220 still produces and transfers the vital signs of body continuously. Obviously, the vital signs at that time become weak or are not deserved to be taken into consideration. Therefore, there is a need of providing an improved vital signs sensing apparatus and an improved method for allowing the monitor patches 220 to output the good quality vital signs.

### SUMMARY

The disclosure is directed to a vital signs sensing apparatus, a sensing method of the vital signs sensing apparatus and a method for processing a vital signs sensing signal.

According to one embodiment, a vital signs sensing apparatus is provided. The vital signs sensing apparatus includes a sound sensing unit and a pressure unit. The sound sensing unit senses a sound inside a body of a user and produces an audio signal. The pressure unit produced a pressure signal. The pressure signal indicates a degree of closeness between the vital signs sensing apparatus and the user. The audio signal is transformed into a processed audio signal according to the pressure signal.

According to another embodiment, a sensing method of a vital signs sensing apparatus is provided. The vital signs sensing apparatus includes a sound sensing unit producing an audio signal and a pressure unit producing a pressure signal. The sensing method includes the following steps. Firstly, the pressure signal is detected, and a force exerted on the pressure unit is determined according to the pressure signal. If the force is smaller than a first strength, it means that the vital signs sensing apparatus is loosened. If the force is larger than the first strength, the audio signal is processed according to the pressure signal and producing a processed audio signal.

According to still another embodiment, a method for processing a vital signs sensing signal is provided. The method comprises the following steps. A detecting mechanism is provided for detecting whether a vital signs sensing apparatus is in close contact with a human body. If the vital signs sensing apparatus is not in close contact with the human body, the vital signs sensing apparatus enters a power-saving state or issues a warning message. If the vital signs sensing apparatus is in close contact with the human body, a degree of closeness between the vital signs sensing apparatus and the human body is detected by the detecting mechanism, the vital signs sensing signal is optimally processed according the degree of closeness, and a processed audio signal outputted.

Numerous objects and features of the disclosure will be readily apparent upon a reading of the following detailed description of embodiments of the invention when taken in conjunction with the accompanying drawings. However, the drawings employed herein are for the purpose of descriptions and should not be regarded as limiting.

### BRIEF DESCRIPTION OF THE DRAWINGS

The above objects and features of the disclosure will become more readily apparent to those ordinarily skilled in the art after reviewing the following detailed description and accompanying drawings, in which:

FIG. 1 (related art) is a schematic diagram of a conventional electrical stethoscope;

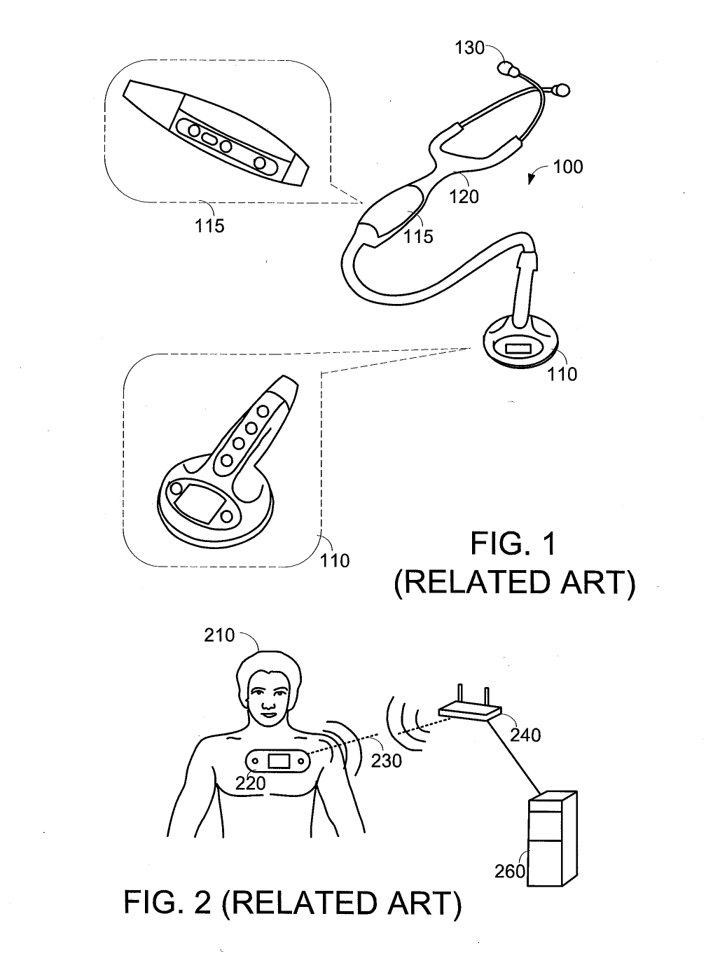

FIG. 2 (related art) is a schematic diagram of a system and method for tracking a vital-signs monitor patch;

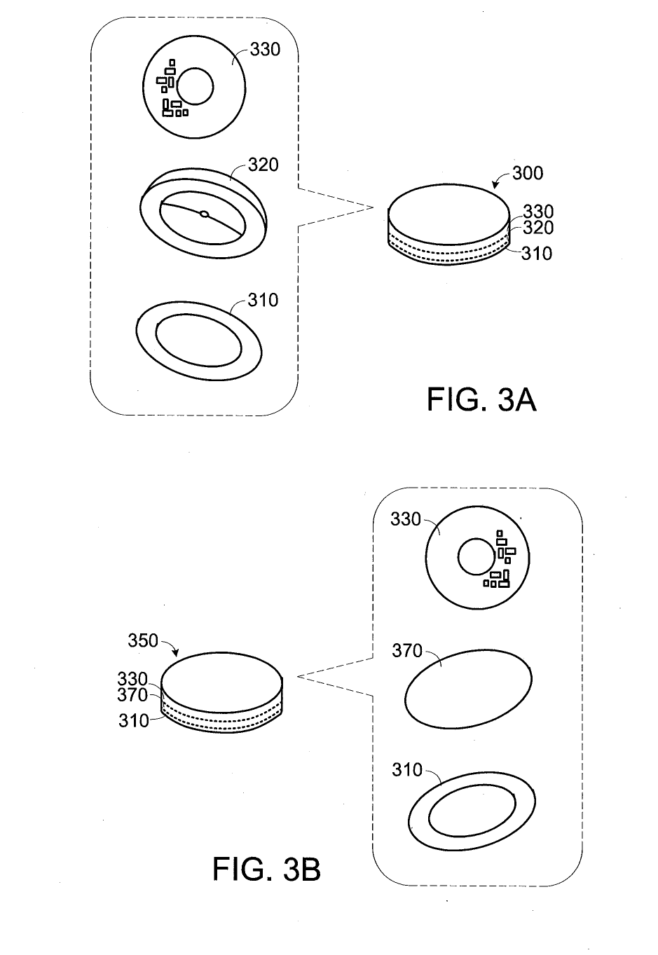

FIG. 3A and FIG. 3B respectively illustrates two embodiments of the vital signs sensing apparatus of the disclosure;

FIG. 4A to FIG. 4C are schematic diagrams of the operation flow of the vital signs sensing apparatus of the disclosure; and

FIG. 5A and FIG. 5B illustrate a block diagram of a sensing method of the vital signs sensing apparatus of the disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION OF PREFERRED EMBODIMENTS

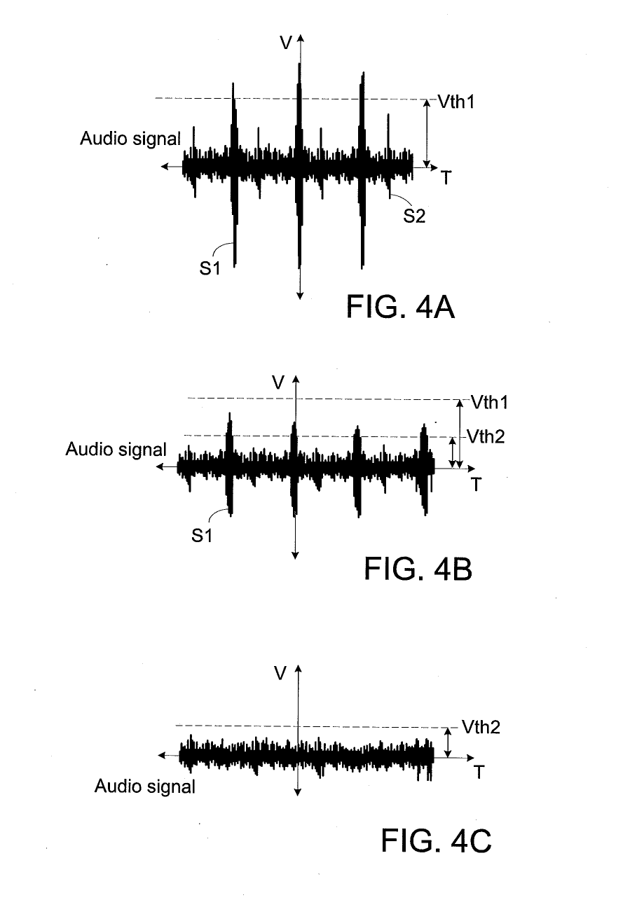

FIG. 3A illustrates one embodiment of a vital signs sensing apparatus of the disclosure. The vital signs sensing apparatus 300 comprises a pressure unit 310, a sound sensing unit 320 and a processing circuit 330. The sound sensing unit 320 is used for sensing the sounds inside the human body. The sound includes for example heart sounds, lung sounds, bowel sounds, fetal heart tones, joint sounds, and etc. And the sound sensing unit 320 produces an audio signal according to the sensed sounds. The processing circuit 330 is connected to the sound sensing unit 320 for processing the audio signal that is produced by the sound sensing unit 320. Afterwards, the processed audio signal is transferred to a backend recording/displaying device (not shown) outside the vital signs sensing apparatus 300 in a wired or wireless transmission manner. Basically, the sound sensing unit 320 may be a condenser microphone, an electret microphone or a dynamic microphone for transforming the sound inside the human body to the audio signal according to the cavity vibration of the sound sensing unit 320.

FIG. 3B illustrates another embodiment of a vital signs sensing apparatus of the disclosure. The vital signs sensing apparatus 350 comprises a pressure unit 310, a sound sensing unit 370 and a processing circuit 330. Compared to the embodiment of FIG. 3A, the difference between these two embodiments is that the sound sensing unit 370 in FIG. 3B is an electroacoustic transducer. The operations of other elements are similar to those of the embodiment of FIG. 3A, and there is no further detailed description here.

In the embodiment of FIG. 3A, the vital signs sensing apparatus 300 comprises a pressure unit 310. The pressure unit 310 produces a pressure signal according to a degree of closeness between the vital signs sensing apparatus 300 and the user. The pressure unit 310 is connected to the processing circuit 330. The processing circuit 330 processes the audio signal according to the pressure signal, and outputs the processed audio signal.

Basically, the resistance of the pressure unit 310 varies with the force exerted on the pressure unit 310. For example, when the magnitude of the force exerted on the pressure unit 310 increases, the resistance decreases. On the other hand, when the magnitude of the force exerted on the pressure unit 310 decreases, the resistance increases. In other words, the resistance of the pressure unit 310 may reflect the pressure signal. The processing circuit 330 can detect the level of the pressure signal and determine the degree of closeness between the user and the pressure unit 310 of the vital signs sensing apparatus 300. According to the embodiment of the disclosure, the pressure unit 310 is a piezoresistive film, a piezoelectric film or a flexible diaphragm.

Of course, the pressure signal is not limited to the resistance of the pressure unit 310. However, those skilled in the art will readily observe that numerous modifications and alterations may be made while retaining the teachings of the disclosure. For example, a constant current may be transmitted from the processing circuit 330 to the pressure unit 310, and the voltage value produced by the pressure unit 310 may reflect the pressure signal. For example, when the magnitude of the pressure applied to the pressure unit 310 increases, the voltage value produced by the pressure unit 310 decreases. On the other hand, when the magnitude of the pressure applied to the pressure unit 310 decreases, the voltage value produced by the pressure unit 310 increases.

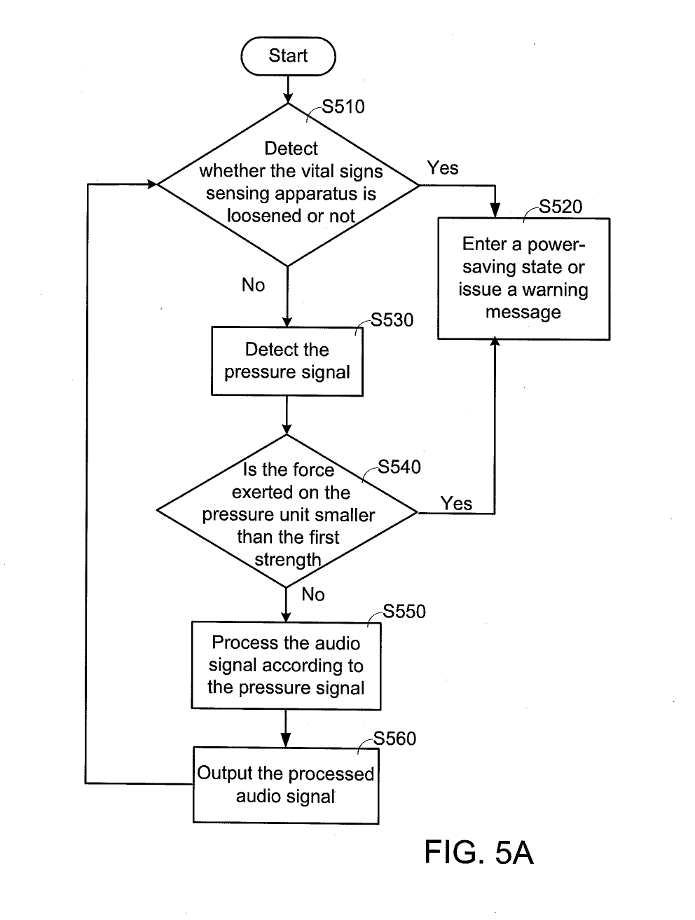

The operation flow of the vital signs sensing apparatus of the disclosure will be illustrated by detecting heart sounds as an example. FIG. 4A to FIG. 4C are schematic diagrams of the operation flow of the vital signs sensing apparatus of the disclosure, wherein a second strength is larger than a first strength. As shown in FIG. 4A, when the vital signs sensing apparatus 300 is in close contact with the user, it can be found that the force exerted on the pressure unit 310 is larger than the second strength according to the pressure signal. At this time, the amplitude of the audio signal of the heart sound is larger, and a first heart sound S1 and a second heart sound S2 can be clearly distinguished. Therefore, the processing circuit 330 detects the first heart sound S1 of the audio signal with a first threshold Vth1 and calculates the heartbeat number of the user.

As shown in FIG. 4B, when the vital signs sensing apparatus 300 is not in close contact with the user, it can be found that the force exerted on the pressure unit 310 is larger than the first strength but smaller than the second strength. At this time, the amplitude of the audio signal of the heart sound is smaller, and only the first heart sound can be distinguished. Therefore, the processing circuit 330 may use a lower second threshold Vth2 to detect the first heart sound S1 of the audio signal. Under this circumstance, the heartbeat number of the user can still be calculated successfully.

As shown in FIG. 4C, when the vital signs sensing apparatus 300 is loosened from the user, it can be found that the force exerted on the pressure unit 310 is smaller than the first strength. At this time, the first heart sound and the second sound of the audio signal cannot be distinguished. Therefore, the processing circuit 330 is unable to successfully calculate the heartbeat number of the user with the second threshold Vth2.

Of course, the above embodiment of changing the threshold value to detect the audio signal is presented herein for purpose of illustration and description only. For the person skilled in the technical field can get the same result by using the processing circuit 330 to provide different gains.

For example, if the force exerted on the pressure unit 310 is larger than the second strength according to the pressure signal, the processing circuit 330 will enlarge the audio signal with a first gain. Then, the processing circuit 330 detects the first heart sound of the audio signal with a threshold and calculates the heartbeat number of the user successfully. If the force exerted on the pressure unit 310 is in the range between the first strength and the second strength according to the pressure signal, the processing circuit 330 will enlarge the audio signal with a second gain, wherein the second gain is larger than the first gain. Then, the processing circuit 330 detects the first heart sound of the audio signal by the same threshold and calculates the heartbeat number of the user successfully. Besides, if the force exerted on the pressure unit 310 is smaller than the first strength, the processing circuit 330 verifies that the vital signs sensing apparatus 300 is loosened from the user, and fails to calculate the heartbeat number of the user successfully.

Furthermore, the processing circuit 330 may use spectrum analysis to determine whether the vital signs sensing apparatus 300 correctly calculates the heartbeat number of the user or not or whether the vital signs sensing apparatus 300 is loosened from the user or not.

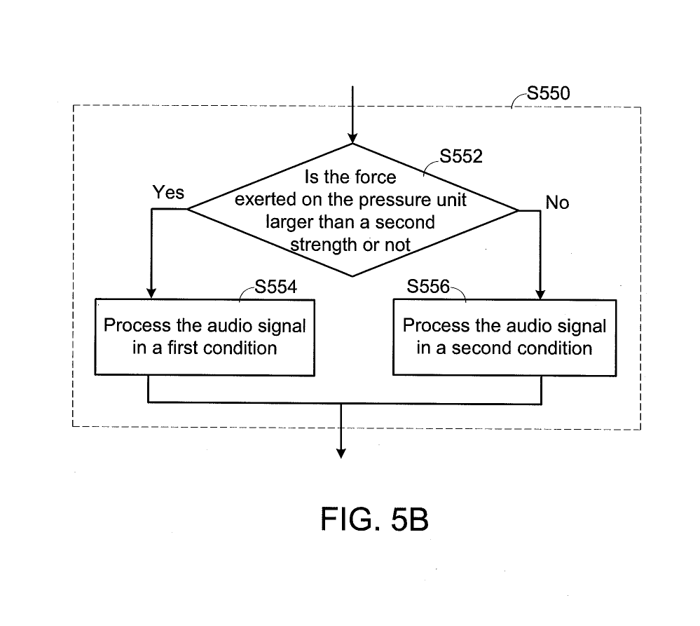

Refer to FIG. 5A and FIG. 5B, which show a block diagram of a sensing method of the vital signs sensing apparatus of the disclosure. When the vital signs sensing apparatus 300 is enabled, the step S510 is firstly performed to detect whether the vital signs sensing apparatus 300 is loosened or not (Step S510). If the vital signs sensing apparatus 300 is loosened, the vital signs sensing apparatus 300 enters a power-saving state or issues a warning message (Step S520). Otherwise, the vital signs sensing apparatus 300 detects the pressure signal when confirming the vital signs sensing apparatus is not loosened (Step S530).

If the vital signs sensing apparatus 300 confirms that the force exerted on the pressure unit is smaller than the first strength (Step S540), it means that the vital signs sensing apparatus 300 is loosened, and then the step S520 is repeatedly done. Otherwise, if the vital signs sensing apparatus 300 confirms that the force exerted on the pressure unit is larger than the first strength (Step S540), the audio signal is processed by the vital signs sensing apparatus 300 according to the pressure signal (Step S550). Afterwards, the processed audio signal is outputted (Step S560) and the step S510 is repeatedly done.

Please refer to FIG. 5B again. The step S550 of processing the audio signal by the vital signs sensing apparatus 300 according to the pressure signal may comprise a step of determining whether the force exerted on the pressure unit 310 is larger than a second strength or not (Step S552).

If the force exerted on the pressure unit 310 is larger than the second strength, it means that the vital signs sensing apparatus 300 is in close contact with the user. Under this circumstance, the audio signal is processed in a first condition (Step S554). That is, the first threshold Vth1 is used to detect the audio signal in order to acquire the heartbeat number of the user.

On the contrary, if the force exerted on the pressure unit 310 is smaller than the second strength, it means that the vital signs sensing apparatus 300 is not in close contact with the user. Under this circumstance, the audio signal is processed in a second condition (Step S556). That is, the second threshold Vth2 is used to detect the audio signal in order to acquire the heartbeat number of the user.

From the above descriptions, the disclosure provides a vital signs sensing apparatus. The force exerted on the pressure unit may be realized according to the pressure signal. In addition, the force exerted on the pressure unit may be used to judge whether the vital signs sensing apparatus is in close contact with the user or the vital signs sensing apparatus is loosened from the user. According to the judging result, the audio signal outputted from the sound sensing unit is correspondingly adjusted. Consequently, the vital signs outputted from the vital signs sensing apparatus have good quality.

Moreover, in the processing method of the vital signs sensing apparatus of the disclosure, a detecting mechanism is used for detecting whether the vital signs sensing apparatus is in close contact with a human body. Of course, in addition to the use of the pressure signal from the pressure unit to determine the degree of closeness, a ratio between the vital signs sensing signal and the background noise may be cooperatively used for providing more accurate judgment. Alternatively, the signal cycle of the vital signs sensing signals may be cooperatively used for providing more accurate judgment. When the vital signs sensing apparatus is loosened from the human body, the vital signs sensing apparatus will enter the power-saving state.

On the contrary, when the vital signs sensing apparatus is in close contact with the human body, the detecting mechanism may further confirm the degree of closeness between the vital signs sensing apparatus and the human body. Next, the vital signs sensing signal is optimally processed according to the degree of closeness. For example, the threshold voltage value or the gain value is properly adjusted. Afterwards, the processed audio signal is outputted.

Furthermore, the vital signs sensing apparatus of the present d can further distinguish the vital signs of a type of the heart sounds, the lung sounds, the bowel sounds, the fetal heart tones and the joint sounds from the envelope signal of the vital signs sensing signal. Besides, the user may manually set the vital signs sensing signal as one of the vital signs of the heart sounds, the lung sounds, the bowel sounds, the fetal heart tones and the joint sounds.

In addition, according to the embodiment of the disclosure, the vital signs sensing apparatus comprises the pressure unit, the sound sensing unit and the processing circuit. However, the components are presented herein for purpose of illustration and description only. The person skilled in the art may use only the pressure unit and the sound sensing unit to constitute the vital signs sensing apparatus. Under this circumstance, the processing circuit is included in the external processing/recording/displaying device.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A vital signs sensing apparatus, comprising:
a sound sensing unit for sensing a sound inside a body of a user and producing an audio signal; and
a pressure unit for producing a pressure signal, wherein the pressure signal indicates a degree of closeness between the vital signs sensing apparatus and the user,
wherein the audio signal is transformed into a processed audio signal according to the pressure signal.

2. The vital signs sensing apparatus according to claim 1, further comprising a processing circuit, wherein the processing circuit is connected to the sound sensing unit and the pressure unit for receiving the audio signal and the pressure signal, and the processing circuit transforms the audio signal into the processed audio signal according to the pressure signal.

3. The vital signs sensing apparatus according to claim 1, wherein the pressure signal is a resistance, a voltage value or a current value.

4. The vital signs sensing apparatus according to claim 1, wherein the sound sensing unit is an electroacoustic transducer, a condenser microphone, an electret microphone or a dynamic microphone.

5. The vital signs sensing apparatus according to claim 1, wherein the pressure unit is a piezoresistive film, a piezoelectric film or a flexible diaphragm.

6. A sensing method of a vital signs sensing apparatus, the vital signs sensing apparatus comprising a sound sensing unit producing an audio signal and a pressure unit producing a pressure signal, the sensing method comprises steps of:
(a) detecting the pressure signal, and determining a force exerted on the pressure unit according to the pressure signal;
(b) if the force is smaller than a first strength, judging that the vital signs sensing apparatus is loosened; and
(c) if the force is larger than the first strength, processing the audio signal according to the pressure signal and producing a processed audio signal.

7. The sensing method according to claim 6, wherein if the vital signs sensing apparatus is determined to be loosened, the sensing method further comprises a step of controlling the vital signs sensing apparatus to enter a power-saving state or issue a warning message.

8. The sensing method according to claim 6, wherein the step (c) comprises:
(c1) if the force is larger than a second strength, processing the audio signal in a first condition, and producing the processed audio signal; and
(c2) if the force is smaller than the second strength, processing the audio signal in a second condition, and producing the processed audio signal;
wherein the first strength is larger than the second strength.

9. The sensing method according to claim 8, wherein in the first condition, the audio signal is processed according to a first threshold, so that the processed audio signal is produced.

10. The sensing method according to claim 9, wherein in the second condition, the audio signal is processed according to a second threshold, so that the processed audio signal is produced, wherein the first threshold is larger than the second threshold.

11. The sensing method according to claim 8, wherein in the first condition, the audio signal is processed according to a first gain, so that the processed audio signal is produced.

12. The sensing method according to claim 11, wherein in the second condition, the audio signal is processed according to a second gain, so that the processed audio signal is produced, wherein the second gain is larger than the first gain.

13. The sensing method according to claim 6, wherein the sound sensing unit is an electroacoustic transducer, a condenser microphone, an electret microphone or a dynamic microphone.

14. The sensing method according to claim 6, wherein the pressure unit is a piezoresistive film, a piezoelectric film or a flexible diaphragm.

15. A method for processing a vital signs sensing signal, the method comprising steps of:
providing a detecting mechanism for detecting whether a vital signs sensing apparatus is in close contact with a human body;
if the vital signs sensing apparatus is not in close contact with the human body, allowing the vital signs sensing apparatus to enter a power-saving state or issue a warning message;
if the vital signs sensing apparatus is in close contact with the human body, using the detecting mechanism to detect a degree of closeness between the vital signs sensing apparatus and the human body, optimally processing the vital signs sensing signal according the degree of closeness, and outputting a processed audio signal.

16. The method according to claim 15, wherein the detecting mechanism for detecting whether the vital signs sensing apparatus is in close contact with the human body is determined according to a ratio between the vital signs sensing signal and a background noise.

17. The method according to claim 15, wherein the detecting mechanism for detecting whether the vital signs sensing apparatus is in contact with the human body is determined according to a signal cycle of the vital signs sensing signal.

18. The method according to claim 15, wherein the detecting mechanism for detecting whether the vital signs sensing apparatus is in close contact with the human body is determined according to a pressure signal outputted from a pressure unit.

19. The method according to claim 15, wherein the audio signal is a heart sound, a lung sound, a bowel sound, a fetal heart tone or a joint sound, wherein a type of the vital signs sensing signal is automatically sensed according to an envelope signal of the vital signs sensing signal, or type of the vital signs sensing signal is manually set.
